(19) United States
(12) Patent Application Publication
(10) Pub. No.: US 2020/0096519 A1
(43) Pub. Date: Mar. 26, 2020

(54) MASS SPECTROMETRY PROFILING OF ESOPHAGEAL CANCER MARKERS

(71) Applicants: Erin Heather SEELEY, Morgantown, WV (US); Katy Ryan Smoot, Morgantown, WV (US); Heather Rae Anderson, Morgantown, WV (US); Steven H. Lin, Houston, TX (US)

(72) Inventors: Erin Heather SEELEY, Morgantown, WV (US); Katy Ryan Smoot, Morgantown, WV (US); Heather Rae Anderson, Morgantown, WV (US); Steven H. Lin, Houston, TX (US)

(21) Appl. No.: 16/574,821

(22) Filed: Sep. 18, 2019

Related U.S. Application Data
(60) Provisional application No. 62/734,570, filed on Sep. 21, 2018.

(51) Int. Cl. G01N 33/68; G01N 1/30; G01N 1/40; H01J 49/16; H01J 49/04

(52) U.S. Cl. CPC G01N 33/6851; G01N 1/30; G01N 1/4044; G01N 2001/302; H01J 49/0463; G01N 2800/52; H01J 49/164

(57) ABSTRACT

This disclosure relates to the use of Matrix-assisted laser desorption ionization (MALDI) mass spectrometry imaging (MSI) to assay tissue samples. Methods of analyzing a tissue sample may generally comprise generating sample ions directly from the tissue sample using a MALDI ionization source, receiving the ions into a mass spectrometer, identifying at least one esophageal tumor related compound in the sample from results from the mass spectrometer, comparing the at least one identified esophageal tumor related compound in the sample to one or more known esophageal tumor profiles, and identifying at least one condition related to the sample from the comparison of the at least one identified esophageal tumor related compound to the one or more known esophageal tumor profiles. The mass spectrometer may be a quadrupole mass spectrometer, a time of flight mass spectrometer, an Orbitrap mass spectrometer, or an ion trap mass spectrometer.

### CROSS REFERENCE TO RELATED APPLICATION

The present application claims the benefit under 35 U.S.C. § 119(e) of U.S. Provisional Application No. 62/734,570, filed Sep. 21, 2018, which is hereby incorporated by reference herein in its entirety.

### FIELD OF INVENTION

The present invention relates to mass spectrometry profiling, and in particular, systems and methods of using matrix-assisted laser desorption ionization (MALDI) mass spectrometry imaging (MSI) of tissue samples to identify a pathologic response to chemoradiation.

### BACKGROUND

Matrix-assisted laser desorption ionization (MALDI) mass spectrometry imaging (MSI) may be used to analyze metabolites, peptides and proteins, DNA segments, and lipids directly from tissue sections with spatial fidelity. MALDI MSI may be performed on either fresh frozen or formalin-fixed, paraffin-embedded (FFPE) tissue specimens. MSI may be used to elucidate molecular profiles of different tumor types and grades including brain, oral, lung, breast, gastric, pancreatic, renal, ovarian and prostate cancers. Conventional MSI may suffer from one or more of the following limitations: spatial resolution, mass accuracy, spectral resolution, sensitivity, robustness, reproducibility, requirements for sample preparation, and degree of technical difficulty.

Accordingly, more efficient mass spectrometry imaging systems and methods of making and using the same are desirable.

### DESCRIPTION OF THE DRAWINGS

The present invention described herein may be better understood by reference to the accompanying figures, in which:

FIG. 1 shows a histology guided mass spectrometry profiling workflow according to the present invention.

FIG. 2 shows a comparison of an average spectrum of esophageal cancer tissue, stroma tissue, normal squamous epithelium tissue, and normal gastric tissue obtained using mass spectrometry methods according to the present invention.

FIG. 3 shows results of classification of partial responders including the number of spectra (% of total).

FIGS. 4A and 4B show results of classification of partial responders and non-responders including the classification accuracy (% of total).

FIGS. 5A and 5B show results of classification of partial responders and non-responders including the classification accuracy (% of total).

FIGS. 6A and 6B show results of classification of partial responders and non-responders including the classification accuracy (% of total).

FIG. 7 shows a method for classifying an esophageal cancer tumor sample according to the present invention.

FIG. 8 shows a method for classifying an esophageal cancer tumor sample according to the present invention.

### DETAILED DESCRIPTION

As generally used herein, the articles “the”, “a”, and “an” refer to one or more of what is claimed or described.

As generally used herein, the terms “include”, “includes”, and “including”, “have”, “has”, and “having”, and “characterized by” are meant to be non-limiting.

As generally used herein, the term “about” refers to an acceptable degree of error for the quantity measured, given the nature or precision of the measurements. Typical exemplary degrees of error may be within 20%, 10%, or 5% of a given value or range of values. Alternatively, and particularly in biological systems, the terms “about” refers to values within an order of magnitude, potentially within 5-fold or 2-fold of a given value.

All numerical quantities stated herein are approximate unless stated otherwise. Accordingly, the term “about” may be inferred when not expressly stated. The numerical quantities disclosed herein are to be understood as not being strictly limited to the exact numerical values recited. Instead, unless stated otherwise, each numerical value is intended to mean both the recited value and a functionally equivalent range surrounding that value. At the very least, and not as an attempt to limit the application of the doctrine of equivalents to the scope of the claims, each numerical parameter should at least be construed in light of the number of reported significant digits and by applying ordinary rounding techniques. Notwithstanding the approximations of numerical quantities stated herein, the numerical quantities described in specific examples of actual measured values are reported as measured.

Any numerical range recited in this specification is intended to include all sub-ranges of the same numerical precision subsumed within the recited range. For example, a range of “1.0 to 10.0” or “1.0-10.0” is intended to include all sub-ranges between (and including) the recited minimum value of 1.0 and the recited maximum value of 10.0, that is, having a minimum value equal to or greater than 1.0 and a maximum value equal to or less than 10.0. Any maximum numerical limitation recited in this disclosure is intended to include all lower numerical limitations subsumed therein and any minimum numerical limitation recited in this disclosure is intended to include all higher numerical limitations subsumed therein. Accordingly, Applicants reserve the right to amend this specification, including the claims, to expressly recite any sub-range subsumed within the ranges expressly recited herein.

This disclosure describes various features, aspects, and advantages of various aspects of the invention. It is understood, however, that this disclosure embraces numerous alternative features, aspects, and/or advantages that may be accomplished by combining any of the various features, aspects, and advantages of the various embodiments described herein in any combination or sub-combination that one of ordinary skill in the art may find useful. Such combinations or sub-combinations are intended to be included within the scope of this disclosure. As such, the claims may be amended to recite any features, aspects, and advantages expressly or inherently described in, or otherwise expressly or inherently supported by, this disclosure. Further, any features, aspects, and advantages that may be present in the prior art may be affirmatively disclaimed. Accordingly, this disclosure may comprise, consist of, or consist essentially of or be characterized by one or more of the features, aspects, and advantages described herein.

In the following description, certain details are set forth in order to provide a better understanding of various features, aspects, and advantages the invention. However, one skilled in the art will understand that these features, aspects, and advantages may be practiced without these details and/or in the absence of any details not described herein. In other instances, well-known structures, methods, and/or techniques associated with methods of practicing the various features, aspects, and advantages may not be shown or described in detail to avoid unnecessarily obscuring descriptions of other details of the various embodiments.

As generally used herein, the term “proteome” refers to the entire complement of proteins produced by an organism or biological system, including modifications made to particular proteins. The proteome of an organism may vary with time, and may also depend on the various stresses that the organism or biological system undergoes. As generally used herein, the term “proteomic profile” refers to information about the protein content of a sample, as characterized by peaks in its mass spectrum corresponding to biomarkers such as proteins, glycoproteins, glycopeptides, peptidoglycans, and other biological substances making up the proteome. The proteomic profile may include all or part of the information, for example, mass spectrometric data, such as m/z values of peaks in the mass spectrum. The proteomic profile may comprise peptide peaks from enzymatic digestion. For example, the proteomic profile may comprise all or substantially all digested proteins and/or lack the entire complement of proteins. It is also possible that substances that are not proteins may be represented in the mass spectrum, for example carbohydrates or lipo-polysaccharides, as well as endogenous peptides, glycoproteins, glycopeptides, peptidoglycans, and other substances as mentioned above. For the sake of simplicity, however, the term “proteomic profile” and “molecular profile” may be used interchangeably herein and each being represented by the peaks in the mass spectra.

As generally used herein, the terms “subject”, “individual”, and “patient” are used interchangeably herein and refer to any mammalian subject, particularly humans. Other subjects may include cattle, dogs, cats, guinea pigs, rabbits, rats, mice, horses, and so on. In some cases, the methods of the invention may find use in experimental animals, in veterinary application, and in the development of animal models, including, but not limited to, rodents including mice, rats, and hamsters, and primates.

As generally used herein, the term “diagnosis” refers to the determination as to whether a subject is likely affected by a given disease, disorder or dysfunction. The skilled artisan often makes a diagnosis on the basis of one or more diagnostic indicators, i.e., a biomarker, the presence, absence, or amount of which may be indicative of the presence or absence of the disease, disorder or dysfunction.

As generally used herein, the term “prognosis” refers to a prediction of the probable course and outcome of a clinical condition or disease, disorder or dysfunction or treatment thereof. A prognosis of a patient is usually made by evaluating factors or symptoms of a disease that are indicative of a favorable or unfavorable course or outcome. For example, a prognosis may be based on one or more diagnostic indicators, i.e., a biomarker, the presence, absence, or amount of which may be indicative of the likely course or outcome of a treatment of a clinical condition or disease, disorder or dysfunction. It is understood that the term “prognosis” does not necessarily refer to the ability to predict the course or outcome with 100% accuracy. Instead, the skilled artisan will understand that the term “prognosis” refers to an increased probability that a certain course or outcome will occur; that is, that a course or outcome is more likely to occur in a patient exhibiting a given condition when compared to patients not exhibiting the condition.

As generally used herein, the term “tumor” refers to any neoplastic cell growth and proliferation, whether malignant or benign, and all pre-cancerous and cancerous cells and tissues. The terms “cancer” and “cancerous” refer to or describe the physiological condition in animals that is typically characterized in part by unregulated cell growth. Cancer refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has typically not penetrated into the lymphatic or blood vessel system or to tissues other than the primary site. The term “′locally advanced cancer” describes non-metastatic cancer that has spread to nearby tissues or lymph nodes but not throughout the body. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but it may also refer to a Stage II cancer or a substage of a Stage II cancer. The skilled artisan will appreciate that the classification of a Stage II cancer as either an early stage cancer or a late stage cancer may depend on the particular type of cancer.

As generally used herein, the terms “sample” refers to a sample that may comprise tumor material obtained from a patient. The term encompasses clinical samples, for example, tissue obtained by surgical resection and tissue obtained by biopsy, such as for example a core biopsy or a fine needle biopsy. The term also encompasses samples comprising tumor cells obtained from sites other than the primary tumor, e.g., metastases and circulating tumor cells, as well as well as preserved samples, such as formalin-fixed, paraffin-embedded samples or frozen samples. The term encompasses cells that are the progeny of the patient's tumor cells, e.g., cell culture samples or cell lines derived from primary tumor cells or circulating tumor cells. The term encompasses samples that may comprise protein or nucleic acid material shed from tumor cells in vivo, e.g., bone marrow, blood, lymph, plasma, serum, and the like. The term also encompasses samples that have been enriched for tumor cells or otherwise manipulated after their procurement and samples comprising polynucleotides and/or polypeptides that are obtained from a patient's tumor material.

As generally used herein, in the context of a cancer, the terms “responsive” and “response” refer to a subject who exhibits or is more likely to exhibit a beneficial clinical response following treatment. By contrast, as generally used herein, the terms “non-responsive” and “non-response” (“NR”) refer to a subject who does not exhibit or is less likely to be exhibit a beneficial clinical response following treatment. As generally used herein, in the context of a cancer, the phrases “partially responsive” and “partial response” refer to a subject who exhibits or is more likely to exhibit a beneficial clinical response following treatment but to a lesser degree than a responsive subject.

As generally used herein, the term “beneficial response” refer to favorable response to a treatment by a subject as opposed to unfavorable responses, i.e., adverse events. For cancer patients, a beneficial response may be expressed in terms of a number of clinical parameters, including loss of detectable tumor (pathologic complete response, “PCR”), decrease in tumor size and/or cancer cell number (pathologic partial response, “PPR”), tumor growth arrest (stable disease, “SD”), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis may be indicative of lack of beneficial response to treatment.

As generally used herein, the term “treatment” refers to administering an agent, or carrying out a procedure (e.g., radiation or a surgical procedure) to obtain a desired pharmacologic and/or physiologic effect. The effect may be prophylactic in terms of completely or partially preventing a disease or symptom thereof and/or may be therapeutic in terms of effecting a partial or complete cure for a disease and/or symptoms of the disease. The effect may be therapeutic in terms of a partial or complete cure for a disease or condition (e.g., a cancer) and/or adverse effect attributable to the disease or condition. These terms also include any treatment of a condition or disease in a mammal, particularly in a human, and include: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; (c) relieving the disease, i.e., causing regression of the disease; (d) reducing the severity of a symptom of the disease and/or (e) reducing the frequency of a symptom of the disease or condition.

Each year in the United States, 17,000 patients are diagnosed with esophageal cancer, which leads to more than 16,000 cancer-related deaths. Adenocarcinoma and squamous cell carcinoma are the two most common types of esophageal cancer, with adenocarcinoma having a higher incidence in the U.S. Worldwide, there are over 450,000 new esophageal cancer cases each year, which lead to more than 400,000 cancer-related deaths. While incidence rates vary from country to country, squamous cell carcinoma is more prevalent worldwide. Currently, the adenocarcinoma form of esophageal cancer is largely attributed to gastroesophageal reflux disease from obesity, and has a 5-year survival rate of 25-30%. The obesity epidemic in developing countries has led to ongoing increases in the incidence of esophageal cancer underscoring urgent health care needs to reduce the incidence of this disease through preventive strategies and, for those who have the disease, to improve the current standard of care for its management. In Asia, smoking and excessive alcohol consumption have led to increased rates of esophageal squamous cell carcinoma. A problem to improving the current standard of care may be the inability for oncologists to accurately predict response to treatment.

For example, conventional diagnostic and imaging approaches may be inadequate to quantify treatment response. The conventional standard of care for locally advanced esophageal cancer is chemoradiation therapy (CRT) followed by an esophagectomy based on clinical trials, which have purportedly established the benefit of preoperative CRT relative to surgery alone. Although cures for this disease may be possible with CRT alone, local recurrence rates of over 50% mandate surgical resection for those patients with incomplete response. However, cure rates for patients with a PCR may exceed 70%. Unfortunately, PCR may be achieved in only 25-30% of patients and may not differ by chemotherapy type or radiotherapy dose (e.g., 41.4-50.4 Gy). This number generally reflects a cure rate of 25% for patients with unresectable disease given CRT alone.

Although adding surgery after CRT may reduce local failures, outcomes from randomized trials have not shown a survival advantage from the addition of surgery owing to high postoperative mortality rates (8-12%). Being able to distinguish a beneficial response, a partial response, and a non-response to CRT may have substantial impacts, including the ability to spare patients with PCR from high-risk surgery. Numerous reports exist on predictive molecular profiles from pretreatment tumor biopsies or clinical nomograms but none have had clinical utility. The most often tested molecular profile is an mRNA expression array, which shows very high predictive value, but has poor reproducibility due to heterogeneity in gene expression and difference in tissue preparations between testing centers. Imaging with barium, esophagogastroduodenoscopy, ultrasonography, and CT may be inadequate for predicting PCR, and even with fluorodeoxyglucose-positron emission tomography (FDG-PET), the area under the curve may at best only 0.77. Conventional imaging techniques are inadequate for predictors of PCR in esophageal cancer due to lack of standardization of these techniques between cancer centers. Accordingly, more accurate predictors of PCR in esophageal cancer may be desirable.

According to the present invention, more efficient and/or cost-effective mass spectrometry imaging systems and methods of making and using the same are described.

The present disclosure describes non-invasive and/or minimally invasive methods that may be used for the screening of esophageal cancer patients for predictive responses to chemoradiation therapy. The methods may include determining whether a proteomic profile of a tumor sample from an esophageal cancer patient includes at least one characteristic predictive of one of a beneficial response, a partial response, and a non-response to treatment include CRT.

The present invention is generally directed to mass spectrometry imaging and analysis that may be useful in cases that are histologically equivocal, and a firm prediction of chemoradiation therapy responsive cells and/or chemoradiation therapy nonresponsive cells cannot be made with absolute certainty. Histology guided mass spectrometry (HGMS) profiling allows for targeted analysis of biomolecules in thin tissue sections from specific cells of interest. HGMS may be used to identify molecular profiles predictive of pathologic response to CRT in esophageal cancer.

Mass spectrometry may be a powerful methodology to detect different forms of a protein because the different forms typically have different masses that may be resolved by the technique (mass spectrometry exploits the intrinsic properties of mass and charge). For example, if one form of a protein is a superior biomarker for a disease over another form of the protein, mass spectrometry may be able to specifically detect and measure the useful form where traditional immunoassay fails to distinguish the forms and/or fails to specifically detect the biomarker.

Mass spectrometry imaging may provide one or more of the following advantages: mass spectrometry works in formalin-fixed paraffin embedded tissue sections; mass spectrometry is a reliable method to differentiate chemoradiation therapy responsive cells and/or chemoradiation therapy nonresponsive cells based on proteomic differences; mass spectrometry may provide information to clarify the diagnosis of ambiguous cases by histology; and mass spectrometry is relatively objective, fast, and relatively inexpensive.

The present invention includes a method of differentiating pathologic responders to chemoradiotherapy from non-responders to chemoradiotherapy using mass spectrometry analysis. Traditional quantitative mass spectrometry has used electrospray ionization (ESI) followed by tandem mass spectrometry (MS/MS), while newer quantitative and qualitative methods are being developed using matrix assisted laser desorption/ionization (MALDI) followed by time of flight (TOF) mass spectrometry (MS).

In ESI tandem mass spectrometry (ESI/MS/MS) analysis of both precursor ions and product ions is possible, thereby monitoring a single precursor product reaction and producing a signal only when the desired precursor ion is present. Protein quantification has been achieved by quantifying tryptic peptides with the use of isotopically labelled peptide standards. Secondary ion mass spectrometry (SIMS) uses ionized particles emitted from a surface for mass spectrometry at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer may be used to measure their mass and charge.

Laser desorption mass spectrometry (LD-MS) involves the use of a pulsed laser, which induces desorption of sample material from a sample site effectively. This method may be used in conjunction with a mass spectrometer and may be performed simultaneously with ionization if one uses the right laser radiation wavelength. When coupled with Time-of-Flight (TOF) measurement, LD-MS is referred to as LDLPMS (Laser Desorption Laser Photoionization Mass Spectrometry). The LDLPMS method of analysis gives instantaneous volatilization (fragmentation) of the sample which permits rapid analysis without any wet extraction chemistry. Signal intensity, or peak height, is measured as a function of travel time. The applied voltage and charge of the particular ion determines the kinetic energy, and separation of fragments is due to the different masses causing different velocities. Each ion mass will thus have a different flight-time to the detector. Other ionization techniques include rapid evaporative ionization mass spectrometry (REIMS) and desorption electrospray ionization (DESI) mass spectrometry

MALDI-TOF mass spectrometry may quantify intact proteins in biological tissue and fluids and is thus applicable to direct analysis of biological tissues and single cell organisms with the aim of characterizing endogenous peptide and protein constituents. Quantification by MALDI-TOF mass spectrometry may be absolute or relative in nature, and it may require the use of internal standards and/or corrections for matrix effects, ionization efficiency, and suppressive effects. The sample is generally mixed with a matrix material which facilitates desorption and ionization of the sample. The mass-to-charge ratio (m/z) is measured as a function of travel time, where separation of the ions is due to the different masses causing different velocities through the drift space and different times of flight to the detector.

Cancer cells and tumors may be characterized by having different proteins, different protein expression levels, and/or different forms of proteins relative to complementary benign cell or tissue samples. Frequently, proteins exist in a sample in a plurality of different forms characterized by detectably different masses. These forms may result from either or both of pre- and post-translational modification. Pre-translational modified forms include allelic variants, splice variants and RNA editing forms. Post-translationally modified forms include forms resulting from proteolytic cleavage (e.g., fragments of a parent protein), glycosylation, phosphorylation, lipidation, oxidation, methylation, cystinylation, sulphonation and acetylation.

When detecting or measuring a protein in a sample, the ability to differentiate between these different forms may depend upon the nature of the difference and the method used to detect or measure the difference. For example, an immunoassay using a monoclonal antibody may detect all forms of a protein containing the epitope and may not distinguish between them. In diagnostic assays, this inability to distinguish different forms of a protein may diminish the power of the assay. Further, only known biomarkers may be targeted. Thus, it is useful to employ an assay method that distinguishes between forms of a protein and that specifically detects and measures a desired form or forms of the protein. Distinguishing different forms of an analyte or specifically detecting a particular form of an analyte is referred to as “resolving” the analyte.

Analysis of chemoradiation therapy responsive cells and/or chemoradiation therapy non-responsive cells using mass spectrometry provides different proteomic profiles. Analysis of the proteomic profiles from chemoradiation therapy responsive cells and/or chemoradiation therapy non-responsive cells may be used to generate proteomic signatures for each, which may be dependent on the type of tissue and the type or severity of the malignancy. These proteomic signatures may then be used to predict or classify an unknown sample. For example, the proteomic profile of an unknown sample may be compared to reference profiles from one or more reference samples to classify the unknown sample as one of a chemoradiation therapy responsive sample, a chemoradiation therapy partially responsive sample, and a chemoradiation therapy non-responsive sample.

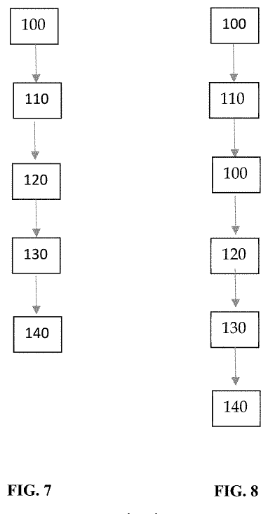

Thus, the present invention provides a method of differentiating esophageal cancer cells likely to be responsive to chemoradiation therapy and esophageal cancer cells that are less likely to be responsive to chemoradiation therapy using mass spectrometry analysis. Referring to FIGS. 7 and 8, the method may generally comprise treating a sample from a patient (100), subjecting the sample from the patient to mass spectrometry (110), obtaining a mass spectrometric proteomic profile from the sample (120), comparing the mass spectrometric proteomic profile to mass spectrometric proteomic profiles of reference samples (130), wherein reference samples include at least one of chemoradiation therapy responsive cells and/or chemoradiation therapy nonresponsive cells, and classifying the sample (140) as one of chemoradiation therapy responsive, chemoradiation therapy partially responsive, and chemoradiation therapy non-responsive based on similarities and/or differences between the mass spectrometric proteomic profile of the sample and the reference sample(s). A chemoradiation therapy responsive sample may be characterized by having no residual viable cancer cells detected. A chemoradiation therapy partially responsive sample may be characterized by having up to 10% viable cancer cells detected. A chemoradiation therapy nonresponsive sample may be characterized by having greater than 10% viable cancer cells detected. The reference sample may comprise a sample from a patient having a known clinical outcome, e.g., chemoradiation therapy responsive or chemoradiation therapy nonresponsive. The reference sample may be used to generate the reference profile. For example, the reference profile may be generated from one or more reference samples, such as at least 10, 25, 50, 100, or more reference samples.

Subjecting the sample from the patient to mass spectrometry (110) may comprise ionizing at least a portion of the sample from the patient using laser energy to generate one or more ions. Obtaining a mass spectrometric proteomic profile from the sample (120) may comprise experimentally measuring the mass to charge ratios of ions to obtain an experimental value corresponding to the mass to charge ratio of each of the ions and distinguishing candidate ions having substantially the same retention time and mass to charge ratio. The method may comprise experimentally measuring the mass to charge ratios of the ions using a quadrupole mass analyzer or a Time of Flight mass analyzer.

For example, the method may comprise conducting MALDI-TOF mass spectrometry on a set of tissue samples from a plurality of patients having esophageal cancer; storing a development set including at least one member having feature values of mass spectrometry data generated as a result of conducting MALDI-TOF mass spectrometry on the set of tissue samples; assigning a classification label to each member of the development set based on whether or not the patient associated with the sample is responsive, partially responsive, or nonresponsive to chemoradiation therapy; separating the development set into at least one of a reference set and a test set; constructing a classifier using one or more of the feature values; filtering the training set by operating on the training set with the classifier and retaining only those members of the training set that meet a performance threshold of classification accuracy; and evaluating the performance of the classifier on the test set. The method may comprise evaluating the performance of the classifier on the reference set including mass spectrometry data of healthy patients and retaining only those members of the reference set having a classification accuracy that meet performance threshold of classification accuracy. The classifier may comprise at least one feature, such as a single feature, a pair of features, or triplets of features in the set of feature values. The performance threshold of classification accuracy may comprise at least 90%, at least 95%, at least 97%, and at least 99%. The at least one feature may comprise a peptide peak.

The mass spectrometric profile may comprise one or more peptide peaks at one or more of 630.41±0.1, m/z 769.48±0.1, m/z 820.48±0.1, m/z 831.53±0.1, m/z 943.60±0.1, m/z 995.56±0.1, m/z 1032.71±0.1, m/z 1157.68±0.1, m/z 1170.69±0.1, m/z 1349.77±0.1, m/z 1684.94±0.1, m/z 1791.97±0.1, and m/z 1850.01±0.1.

Referring to FIGS. 7 and 8, the method may comprise treating the sample prior to and/or after subjecting the sample to mass spectrometry. Treating a sample may comprise at least one of collecting at least one serial section of the sample, staining the sample, subjecting the sample to deparaffinization and antigen retrieval, subjecting the sample to on-tissue tryptic digestion, applying a MALDI compatible matrix to the sample, annotating a digital microscopy image of the reference profile and imposing the annotated digital microscopy image of the reference profile onto an image of the sample profile. For example, the method may comprise subjecting the sample to deparaffinization, subjecting the sample to antigen retrieval, subjecting the sample to on-tissue tryptic digestion, and/or applying a MALDI compatible matrix to the sample prior to subjecting the sample to mass spectrometry. For example, the method may comprise staining the sample prior to subjecting the stained sample to mass spectrometry. In another example, the method may comprise staining the sample after subjecting the sample to mass spectrometry. Annotating the sample may comprise marking targets, or areas of interest on the sample that may have a diameter from 10-400 micrometers, such as 50-100 micrometers and 250-350 micrometers. Subjecting the sample to mass spectrometry may comprise subjecting the targets or areas of interest to mass spectrometry. Subjecting the sample to mass spectrometry may comprise subjecting the entire sample, i.e., targets or areas of interest as well as other areas of the sample, to mass spectrometry.

In another embodiment, annotation of the sample may occur after part of or the entire sample has been subjected to mass spectrometry such that selection of the areas of interest is performed post-acquisition of mass spectral data. In this example, the analyzed sample may be stained, and the digital microscopy image annotated for regions of interest; these regions of interest may be superimposed onto the image of the sample use in the mass spectrometry analysis to isolate their components of the spectral data file. In another example, a serial section of the sample may be stained, and the digital microscopy image annotated for regions of interest; these regions of interest may be superimposed onto the image of the sample use in the mass spectrometry analysis to isolate their components of the spectral data file.

The method may comprise collecting a first serial section of the sample and a second serial section of the sample, staining the first serial section of the sample, and subjecting the second serial section of the sample to mass spectrometry. For example, a first serial section of the sample may be stained and a second serial section may be subjected to mass spectrometry. In other words, the treated sample may not be subjected to mass spectrometry. Instead, the regions of the other one of the serial sections that correspond to the annotated sections of the treated sample may be subjected to mass spectrometry.

Without wishing to be bound to any particular theory, subjecting the sample to mass spectrometry prior to annotation may reduce the reproducibility of results for one or more of the following reasons: exposure to the laser may cause mild to moderate destruction of the sample which may impede proper observation and analysis of the sample for staining and annotation; the use of larger regions of the sample for analysis may encourage less particularity when defining the region for cancer cell analysis; and decreased particularity may result in a wider array of cell types within the sample increasing the variability of results. Limiting the data to the areas of interest may provide for more accurate classification and results as there is less “noise” from inappropriate cell types. Without wishing to be bound to any particular theory, noise, in this context, may include signals originating from other cell types than the ones of interest, such as red blood cells, inflammatory cells, and the like.

The mass spectrometric proteomic profile may include one or more peaks of monoisotopic mass. The area under the peak for the monoisotopic mass may be used to determine the abundance value. The monoisotopic mass is the sum of the masses of the atoms in a molecule using the unbound, ground-state, rest mass of the principal (most abundant) isotope for each element instead of the isotopic average mass. Monoisotopic mass is typically expressed in unified atomic mass units (u), also called Daltons (Da). The mass spectrometric proteomic profile may not include all of the peaks detected from the sample. Without wishing to be bound by theory, it is thought that the peaks represent unique biomarkers that are useful for fast detection and identification of cancer in the samples. These biomarkers do not need to be identified (e.g., myosin) to be used as mass signature components of the mass spectrometric proteomic profile. When samples that have been enzymatically digested as part of the treatment process, more than one peak may be associated with the same biomarker as a result of the fragmentation of the protein parent molecules into constituent sub-protein level biomolecules (e.g. peptides, glycans). For example, one protein biomarker may have several peptides detected and comprising part of the mass spectrometric proteomic profile.

As discussed above, in various embodiments, a prognosis of a treatment of an esophageal cancer patient may be based on characteristic similarities or differences (e.g., proteomic signature) between tumor samples and/or a tumor sample and reference sample. For example, if the proteomic profile is presented in the form of a mass spectrum, the proteomic signature may be a peak or a combination of peaks that differ, qualitatively or quantitatively, from the mass spectrum of a corresponding reference sample. Thus, the appearance of a new peak or a combination of new peaks in the mass spectrum, or any statistically significant change in the amplitude or shape of an existing peak or combination of existing peaks, or the disappearance of an existing peak in the mass spectrum may be considered a proteomic signature for one of a CRT responsive, CRT partially responsive, and CRT non-responsive for a tumor sample.

Statistical methods for comparing proteomic profiles may be defined by the peak amplitude values at key mass/charge (m/z) positions along the horizontal axis of the mass spectrum. A characteristic proteomic profile may, for example, be characterized by the pattern formed by the combination of spectral amplitudes at given m/z vales. The presence or absence of a proteomic signature for one of a CRT responsive, CRT partially responsive, and CRT non-responsive for a tumor sample, or the substantial identity of two proteomic profiles, may be determined by matching the proteomic profile of a tumor sample with the proteomic profile of a reference sample using an appropriate algorithm.

The presently disclosed invention provides improved methods for differentiating between chemoradiation therapy responsive esophageal cancer cells and chemoradiation therapy nonresponsive esophageal cancer cells. More specifically, the present invention includes a targeted approach in which only discrete areas within a tissue sample are analyzed. Histological or immunohistological staining may also be used to guide the acquisition of mass spectrometry imaging so that each area analyzed may be enriched for a single cell type. Such analysis may be more conducive to statistical analysis and classification algorithm generation. Further, such methods may provide a biological insight into the classification which is not attainable through standard histological techniques (i.e., disease outcome, improved diagnostics, treatment responses).

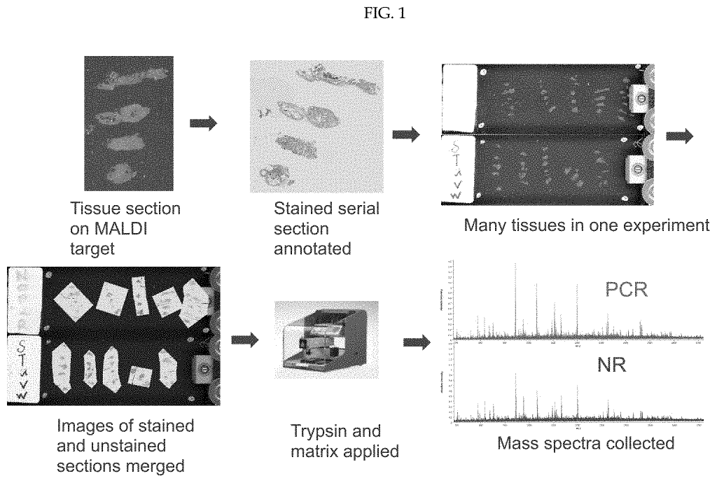

Referring to FIG. 1, a tissue sample is collected onto an indium tin oxide (ITO) coated glass slide that is compatible with a MALDI TOF mass spectrometer. A serial section is collected onto a standard microscopy slide and stained with hematoxylin and eosin (H&E). The stained section is scanned using a digital slide scanner and made available to a pathologist who annotates histological regions of interest within the section. The annotated stained section is registered with a digital image of the serial unstained section using Photoshop® (or equivalent software) allowing for the coordinates of the annotations to be obtained. The unstained section is subjected to sample preparation including deparaffinization, antigen retrieval, tryptic digestion, and matrix application. Mass spectra are collected from the designated locations and the spectra subjected to statistical analysis.

A biomarker in the tissue sample may be modified before analysis to improve its resolution or to determine its identity. For example, the biomarkers may be subject to proteolytic digestion before analysis using a protease. Proteases, such as trypsin, that are likely to cleave the biomarkers into a discrete number of fragments may be particularly useful. Due to their individual characteristics, each fragment will be detected as one or more unique masses and will experience differences in ionization efficiency, resolution, and sensitivity during mass spectrometry analysis. One, multiple, or all fragments of a biomarker may be detected in a single analysis. The fragments that result from digestion function as a fingerprint for the biomarkers, thereby enabling their detection indirectly. This may be particularly useful where there are biomarkers with similar molecular masses that might be confused for the biomarker in question. Also, proteolytic fragmentation may be useful for high molecular weight biomarkers because smaller biomarkers may be more easily resolved and/or detected by mass spectrometry. Typically, the peptide fragments resulting from the enzymatic digestion of a protein biomarker are more easily detected than the original parent protein itself due to improved ionization efficiency and desorption efficiency, leading to improved sensitivity. Additionally, proteolytic digestion allows for measurement of uncrosslinked protein segments from larger proteins that have been crosslinked due to fixation processes. This is particularly useful in FFPE tissues where large protein biomarkers are crosslinked and very difficult to liberate from the tissue surface through desorption ionization processes. Carefully controlled enzymatic digestion treatment that maintains spatial localization increases the sensitivity of detection for the resultant biomarker fragment peptides, glycans, in a robust and reproducible manner. In a histology guided mass spectrometry approach, this sample treatment process results in complex biomarker fragment spectra for each type of cell subpopulation, for which these types of large, robust data sets are well-suited for statistical analysis by machine learning and artificial intelligence platforms. In another example, biomarkers may be modified to improve detection resolution. For instance, neuraminidase may be used to remove terminal sialic acid residues from glycoproteins to improve binding to an anionic adsorbent and to improve detection resolution. In another example, the biomarkers may be modified by the attachment of a tag of particular molecular weight that specifically binds to molecular biomarkers, further distinguishing them.

### EXAMPLES

The various embodiments herein may be better understood when read in conjunction with the following representative examples. The following examples are included for purposes of illustration and not limitation.

### Example 1

Esophageal cancer affects about 17,000 people in the United States each year resulting in about 16,000 deaths. The standard of care is generally treating with chemotherapy and concurrent radiation prior to surgery. About 30% of patients will show a pathologic complete response (PCR) to chemoradiation therapy (CRT). On average, a 10% mortality rate is observed due to complications from surgery. An objective test that may predict PCR or non-response from pretreatment biopsies could help to tailor treatment for an esophageal cancer patient.

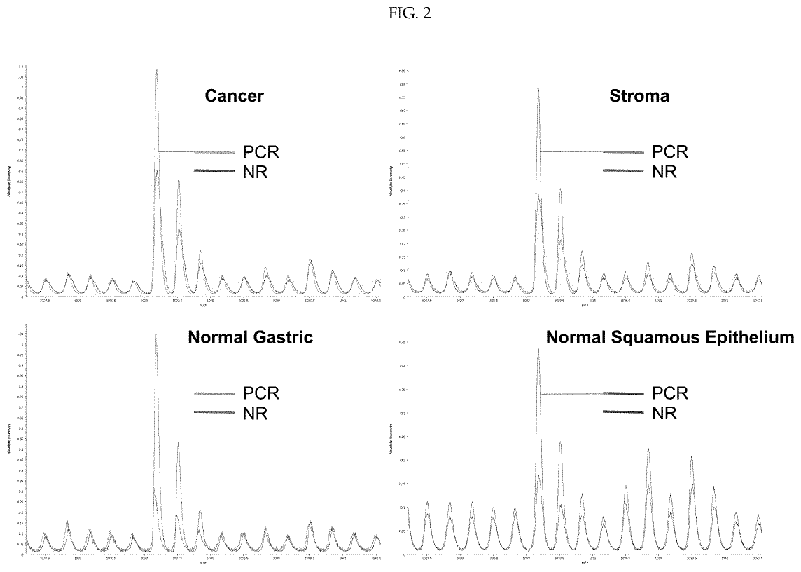

Esophageal biopsy samples were analyzed from 89 total patients, including 46 PCR and 43 non-responders. Areas analyzed included cancer, stroma, normal squamous epithelium, and normal gastric tissue. The data were subjected to statistical analysis and machine learning classification algorithm generation. See Table 1 and FIG. 2.

TABLE 1

| | Peaks with | LDA Internal |
|---|---|---|
| | Significant | Cross Validation |
| Cell Type | p-values | Accuracy |
| Cancer | 391 | 80.2% |
| Stroma | 396 | 77.8% |
| Normal Squamous Epithelium | 187 | 86.9% |
| Normal Gastric | 30 | |

Significant differences in mass spectral profiles were observed between PCR and non-responder patients. The highest classification accuracy was found in normal appearing tissue surrounding tumorous regions. Without wishing to be bound to any particular theory, molecular differences in surrounding healthy tissue may be linked to the overall activity of a patient's immune response to cancerous tissue.

### Example 2

A proteomic signature that correlates with response to chemoradiation and whether partial responders more closely resemble PCR or non-responders was determined using HGMS profiling. Without wishing to be bound to any particular theory, the present invention had about an 87% accuracy in leave-10%-out internal cross validation and the greatest differences were observed in normal-appearing tissue.

For patients with locally advanced esophageal cancer, multimodal therapy consisting of concurrent chemoradiation therapy (CRT) followed by surgery is the standard of care. Benefits to neoadjuvant CRT include tumor downstaging, and it is estimated that nearly one-third of patients undergo a complete pathologic response (PCR) which has been linked to improved overall survival. Predicting pathologic response to CRT may lead to tailored therapy; however, no accurate methods currently exist. HGMS allows for targeted analysis of biomolecules in thin tissue sections from specific cells of interest. HGMS was used to identify molecular profiles predictive of pathologic response to CRT in esophageal cancer.

Referring to FIG. 1, formalin fixed, paraffin embedded (FFPE) biopsies from 47 esophageal cancer patients were treated with concurrent CRT (50.4 Gy in 28 fractions) followed by surgery at about 8 weeks. About 5 μm thick sections were collected onto indium tin oxide (ITO) coated glass slides for HGMS or on regular glass slides for hematoxylin and eosin (H&E) staining. Digital images of stained sections were annotated with areas of tumor, stroma, normal squamous epithelium, and normal gastric tissue. HGMS sections were deparaffinized and antigen retrieved prior to on-tissue tryptic digestion and matrix application using a robotic sprayer. Mass spectra were collected from the annotated regions using MALDI TOF MS. Statistical analysis and classification algorithm generation were performed using SCiLS Lab.

Table 2 shows a summary of the number of samples having each histology and clinical outcome. Percentages indicate the residual viable tumor cells remaining after CRT. Patients were divided into responders, non-responders, or partial responders based on the percentage of viable residual tumor cells.

TABLE 2

| | PCR | Non- | Partial |
|---|---|---|---|
| | 0% | Responders >10% | Responders <1%-10% |
| Cell Type | residual | residual | residual |
| Cancer | 27 | 12 | 8 |
| Stroma | 24 | 12 | 8 |
| Normal | 19 | 6 | 5 |
| Squamous | | | |
| Epithelium | | | |
| Normal Gastric | 3 | 5 | 2 |

Table 3 shows statistical analysis results for comparisons between PCR and non-responders for each histology in which both normal regions were combined to allow for more statistical power for linear discriminant analysis (LDA), a type of machine learning algorithm. Significant p-values indicated that a peak was differentially expressed between groups, receiver operator characteristic (ROC) curves provide a measure of how well a peak may differentiate between groups, internal cross validation provides an estimate of the accuracy of a classification algorithm.

TABLE 3

| | Peaks with | Peaks with | |
|---|---|---|---|
| | Significant | area under ROC | LDA Internal Cross |
| Cell Type | p-values | curve >0.8 or <0.2 | Validation Accuracy |
| Cancer | 78 | 0 | 84.33% |
| Stroma | 109 | 0 | 69.17% |
| Normal | 359 | 10 | 69.23% |
| Squamous | | | |
| Epithelium | | | |
| Normal Gastric | 82 | 24 | |

FIG. 2 shows an example of a peptide that was found to be significantly more abundant in PCR samples in all 4 histological regions studied. The peptide was identified as originating from histone H3.

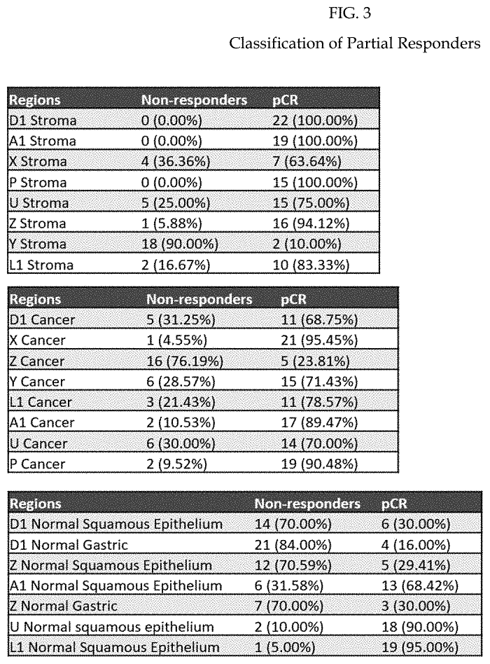

FIG. 3 shows spectra collected from the partial responders which show mixed results. Cancer and stroma tissue favor PCR in most samples while the normal tissue favors non-responders. The classification results from these samples show that they fall in between PCR and non-responders.

Without wishing to be bound to any particular theory, Tables 2 and 3 and FIGS. 2 and 3 show the following: (a) significant differences observed between PCR and non-responder patients; (b) greatest number of significant peaks in normal appearing tissue; and (c) partial responders give mixed results when classified using LDA.

### Example 3

A proteomic signature that correlates with response to chemoradiation and whether partial responders more closely resemble PCR or non-responders was determined using HGMS profiling. Without wishing to be bound to any particular theory, the present invention had about 90% accuracy (about 86% for PCR and about 93% for non-responders) for the HGMS analysis of esophageal cancer cell subpopulations. Other cell types, including high grade dysplasia, normal stroma, and desmoplastic stroma were also evaluated, but the esophageal cancer cell type was found to be the most useful in this example.

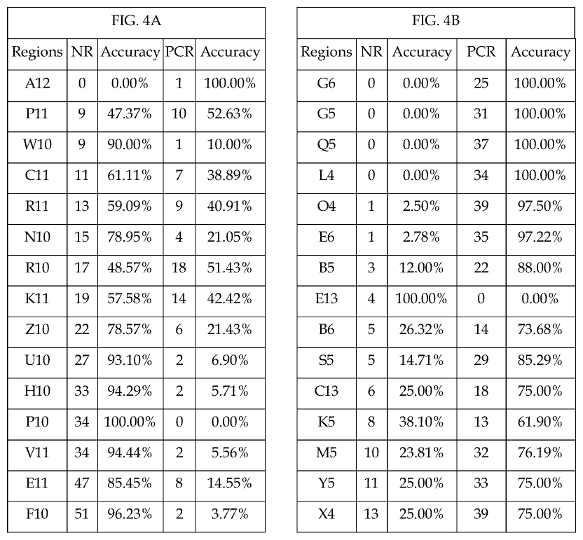

Referring to FIGS. 4A and 4B, the training set included 19 non-responder samples and 20 PCR samples for the analysis of esophageal cancer cell subpopulations, and the validation set included 15 non-responder samples and 15 PCT samples. The training set included at least 13 discriminatory peaks. The non-responder validation set included 14 set in which 64.3% were correctly classified and 35.7% were indeterminate. The PCR validation set included 14 set in which 92.9% were correctly classified and 7.1% were indeterminate. The overall accuracy was 78.6%.

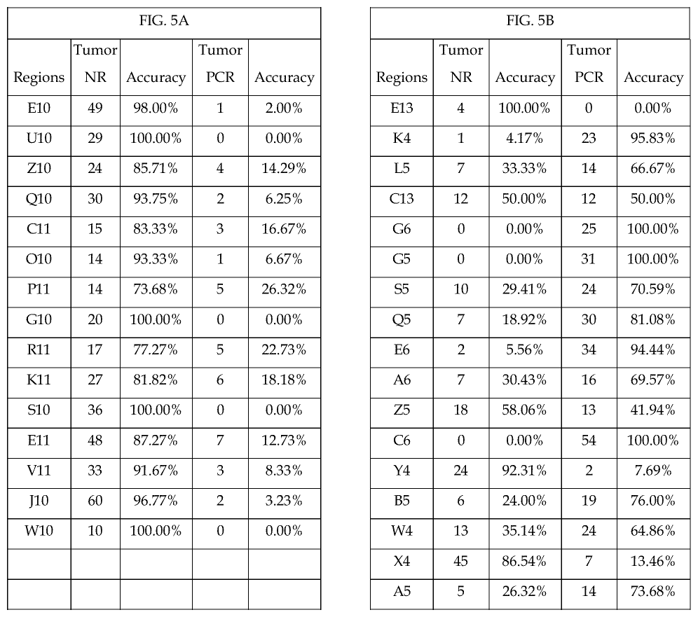

Referring to FIGS. 5A and 5B, the training set included 19 non-responder samples and 18 PCR samples for the analysis of esophageal cancer cell subpopulations, and the validation set included 15 non-responder samples and 17 PCT samples. The training set included at least 13 discriminatory peaks. The non-responder validation set included 15 set in which 100.0% were correctly classified. The PCR validation set included 16 set in which 68.8% were correctly classified, 12.5% were incorrectly classified, and 18.8% were indeterminate. The overall accuracy was 83.9%.

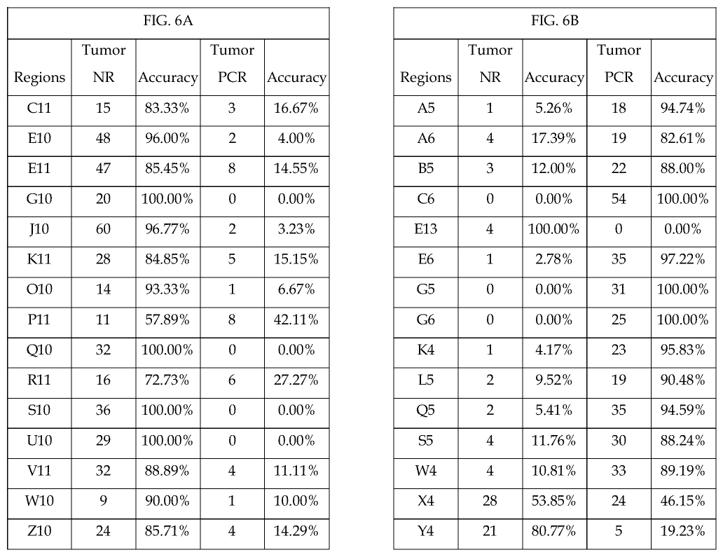

Referring to FIGS. 6A and 6B, the training set included 19 non-responder samples and 20 PCR samples for the analysis of esophageal cancer cell subpopulations, and the validation set included 15 non-responder samples and 15 PCT samples. The training set included at least 13 discriminatory peaks. The non-responder validation set included 15 set in which 93.3% were correctly classified and 6.67% was indeterminant. The PCR validation set included 14 set in which 85.7% were correctly classified, 7.1% were incorrectly classified, and 7.1 were indeterminate. The overall accuracy was 89.7%.

The following aspects are disclosed in this application:

Aspect 1. A method of analyzing a tissue sample comprising: (a) generating sample ions directly from the tissue sample using a MALDI ionization source; (b) receiving the ions into a mass spectrometer; (c) identifying at least one esophageal tumor related compound in the sample from results from the mass spectrometer; (d) comparing the at least one identified esophageal tumor related compound in the sample to one or more known esophageal tumor profiles; and (e) identifying at least one condition related to the sample from the comparison of the at least one identified esophageal tumor related compound to the one or more known esophageal tumor profiles.

Aspect 2. A method for analyzing an esophageal tumor sample from a subject characterized as at least one of chemoradiation therapy responsive, chemoradiation therapy partially responsive, and chemoradiation therapy nonresponsive, the method comprising: (a) treating the sample; (b) subjecting the sample to mass spectrometry; (c) generating a mass spectrometry profile from the sample; (d) comparing the mass spectrometry profile of the sample to a reference profile, wherein the reference profile includes a statistical average profile from a plurality of chemoradiation therapy responsive esophageal tumor samples, chemoradiation therapy partially responsive esophageal tumor samples and/or chemoradiation therapy nonresponsive esophageal tumor samples; and (e) classifying the esophageal cancer sample as at least one of chemoradiation therapy responsive, chemoradiation therapy partially responsive, and/or chemoradiation therapy nonresponsive esophageal tumor samples based on a level of similarity between the mass spectrometry profile of the sample and the reference profile.

Aspect 3. The method according to one or more of the foregoing aspects, wherein the mass spectrometry is matrix-assisted laser desorption mass spectrometry.

Aspect 4. The method according to one or more of the foregoing aspects, wherein treating the sample comprises preparing the sample for mass spectrometry selected from: (a) staining the sample; (b) subjecting the sample to deparaffinization and antigen retrieval prior to on-tissue tryptic digestion and matrix application using a robotic sprayer; (c) subjecting the sample to on-tissue tryptic digestion; and (d) applying a MALDI compatible matrix.

Aspect 5. The method according to one or more of the foregoing aspects, wherein treating the sample comprises: (a) annotating a digital microscopy image of the reference profile; and (b) imposing the annotated digital microscopy image of the reference profile upon the sample prior to mass spectrometry.

Aspect 6. The method according to one or more of the foregoing aspects, wherein treating the sample is performed before subjecting the sample to mass spectrometry.

Aspect 7. The method according to one or more of the foregoing aspects, wherein treating the sample is performed after subjecting the sample to mass spectrometry.

Aspect 8. The method according to one or more of the foregoing aspects, wherein subjecting the sample to mass spectrometry and obtaining a mass spectrometry profile from the samples are performed before treating the sample.

Aspect 9. The method according to one or more of the foregoing aspects, wherein the treatment comprises: (a) staining the sample; and (b) annotating a digital microscopy image of the sample.

Aspect 10. The method according to one or more of the foregoing aspects, wherein the reference profile is generated using a machine learning algorithm which defines one or more biomarkers.

Aspect 11. The method according to one or more of the foregoing aspects, wherein the reference profile comprises biomarkers represented by at one of at least 50, at least 100, at least 250, and at least 500 peptide peaks having unique m/z values.

Aspect 12. The method according to one or more of the foregoing aspects, wherein the reference profile or sample is stained with hematoxylin and eosin.

Aspect 13. The method according to one or more of the foregoing aspects, comprising repeating the steps of (a)-(e) on the sample.

Aspect 14. The method according to one or more of the foregoing aspects, comprising treating the patient with at least one of chemotherapy, immunotherapy, toxin therapy, surgery, and radiotherapy when the patient is identified as having chemoradiation therapy treatment responsive, chemoradiation therapy partially responsive and/or nonresponsive esophageal cells.

Aspect 15. The method according to one or more of the foregoing aspects, comprising performing histologic analysis on the esophageal cancer sample.

Aspect 16. The method according to one or more of the foregoing aspects, wherein the plurality of known pathologic complete responders to chemoradiotherapy, the plurality of known partial responders to chemotherapy, and the plurality of known non-responders to chemoradiotherapy are identified by immunohistochemical analysis, genetic analysis, patient outcome, or a combination thereof.

Aspect 17. The method according to one or more of the foregoing aspects, comprising immunohistochemical analysis on the esophageal cancer sample.

Aspect 18. The method according to one or more of the foregoing aspects, wherein the method has a sensitivity and a specificity of at least 75% in correctly classifying the esophageal cancer sample.

Aspect 19. The method of according to one or more of the foregoing aspects, wherein the statistical average profile comprises at least one of m/z 630.41±0.1, m/z 769.48±0.1, m/z 820.48±0.1, m/z 831.53±0.1, m/z 943.60±0.1, m/z 995.56±0.1, m/z 1032.71±0.1, m/z 1157.68±0.1, m/z 1170.69±0.1, m/z 1349.77±0.1, m/z 1684.94±0.1, m/z 1791.97±0.1, and m/z 1850.01±0.1.

Aspect 20. A method for screening an esophageal cancer patient for a predictive response to chemoradiation therapy, the method comprising: (a) treating the sample; (b) subjecting a sample from a patient to mass spectrometry; (c) obtaining a mass spectrometry profile from the sample; (d) comparing the mass spectrometry profile of the sample to a reference profile, wherein the reference profile includes a statistical average profile from a plurality of chemoradiation therapy responsive esophageal tumor samples, chemoradiation therapy partially responsive, and/or chemoradiation therapy nonresponsive esophageal tumor samples; and (e) classifying the sample as at least one of chemoradiation therapy responsive, chemoradiation therapy partially responsive, and/or chemoradiation therapy nonresponsive esophageal tumor samples based on a level of similarity between the mass spectrometry profile of the esophageal cancer sample and the reference profile.

Aspect 21. A method of analyzing a tissue sample comprising: (a) generating sample ions directly from the tissue sample using a MALDI ionization source; (b) receiving the ions into a mass spectrometer; (c) identifying at least one esophageal tumor related compound in the tissue sample from results from the mass spectrometer; (d) comparing the at least one identified esophageal tumor related compound in the tissue sample to one or more known esophageal tumor profiles; and (e) identifying at least one condition related to the tissue sample from the comparison of the at least one identified esophageal tumor related compound to the one or more known esophageal tumor profiles.

Aspect 22. The method according to one or more of the foregoing aspects, wherein the tissue sample comprises an esophageal tumor sample from a subject characterized as at least one of chemoradiation therapy responsive, chemoradiation therapy partially responsive, and chemoradiation therapy nonresponsive.

Aspect 23. The method according to one or more of the foregoing aspects, wherein the one or more known esophageal tumor profiles comprise at least one of a profile from a plurality of chemoradiation therapy responsive esophageal tumor samples, a profile from a plurality of chemoradiation therapy partially responsive esophageal tumor samples, and a profile from a plurality of chemoradiation therapy nonresponsive esophageal tumor samples.

Aspect 24. The method according to one or more of the foregoing aspects, wherein the one or more known esophageal tumor profiles comprise at least one of m/z 630.41±0.1, m/z 769.48±0.1, m/z 820.48±0.1, m/z 831.53±0.1, m/z 943.60±0.1, m/z 995.56±0.1, m/z 1032.71±0.1, m/z 1157.68±0.1, m/z 1170.69±0.1, m/z 1349.77±0.1, m/z 1684.94±0.1, m/z 1791.97±0.1, and m/z 1850.01±0.1.

Aspect 25. The method according to one or more of the foregoing aspects, wherein the at least one condition comprises at least one of chemoradiation therapy responsive, chemoradiation therapy partially responsive, and chemoradiation therapy nonresponsive.

Aspect 26. The method according to one or more of the foregoing aspects, wherein identifying at least one esophageal tumor related compound in the sample from results from the mass spectrometer comprises generating a mass spectrometry profile of the tissue sample from results from the mass spectrometer.

Aspect 27. The method according to one or more of the foregoing aspects, wherein the mass spectrometry profile comprises at least one of m/z 630.41±0.1, m/z 769.48±0.1, m/z 820.48±0.1, m/z 831.53±0.1, m/z 943.60±0.1, m/z 995.56±0.1, m/z 1032.71±0.1, m/z 1157.68±0.1, m/z 1170.69±0.1, m/z 1349.77±0.1, m/z 1684.94±0.1, m/z 1791.97±0.1, and m/z 1850.01±0.1.

Aspect 28. The method according to one or more of the foregoing aspects characterized by a sensitivity and a specificity of at least 95% in correctly classifying the tissue sample as one of cancerous and non-cancerous.

Aspect 29. The method according to one or more of the foregoing aspects comprising staining the tissue sample prior to generating sample ions directly from the tissue sample using a MALDI ionization source.

Aspect 30. The method according to one or more of the foregoing aspects comprising staining the tissue sample after generating sample ions directly from the tissue sample using a MALDI ionization source.

Aspect 31. The method according to one or more of the foregoing aspects comprising staining the sample with hematoxylin and eosin.

Aspect 32. The method according to one or more of the foregoing aspects comprising: treating the tissue sample to deparaffinization and antigen retrieval prior to on-tissue tryptic digestion and matrix application using a robotic sprayer; treating the tissue sample to on-tissue tryptic digestion; and applying a MALDI compatible matrix to the treated tissue sample using a robotic sprayer.

Aspect 33. The method according to one or more of the foregoing aspects, wherein the tissue sample comprises a first section and a second section, and wherein the method comprising annotating an image of the first section to identify areas of interest on the tissue sample having a diameter from 10-400 micrometers; imposing the annotated image of the first section upon the second section prior to generating sample ions directly from the areas of interest identified on the second section of the tissue sample using the MALDI ionization source.

Aspect 34. The method according to one or more of the foregoing aspects, wherein the areas of interest are identified as one of cancerous and non-cancerous using histologic analysis and/or immunohistochemical analysis.

Aspect 35. The method according to one or more of the foregoing aspects, wherein the tissue sample is obtained from a patient.

Aspect 36. The method according to one or more of the foregoing aspects comprising treating the patient with at least one of chemotherapy, immunotherapy, toxin therapy, surgery, and radiotherapy when the patient is identified as having at least one of a chemoradiation therapy responsive condition, a chemoradiation therapy partially responsive condition, and a chemoradiation therapy nonresponsive condition.

Aspect 37. A method of treating an esophageal tumor in a patient comprising: (i) determining chemoradiation responsive condition in the patient by (a) obtaining a tissue sample from the patient; (b) generating sample ions directly from the patient's sample using a MALDI ionization source; (c) receiving the ions into a mass spectrometer; (d) identifying at least one esophageal tumor related compound in the tissue sample from results from the mass spectrometer; and (ii) administering at least one of chemotherapy, immunotherapy, toxin therapy, surgery, and radiotherapy to the patient to treat the esophageal tumor when the patient is identified as one of chemoradiation therapy responsive, chemoradiation therapy partially responsive and chemoradiation therapy nonresponsive.

Aspect 38. The method according to one or more of the foregoing aspects comprising (e) comparing the at least one identified esophageal tumor related compound in the tissue sample to one or more known esophageal tumor profiles; and (f) identifying at least one condition related to the tissue sample from the comparison of the at least one identified esophageal tumor related compound to the one or more known esophageal tumor profiles.

Aspect 39. The method according to one or more of the foregoing aspects, wherein the one or more known esophageal tumor profiles comprise at least one of m/z 630.41±0.1, m/z 769.48±0.1, m/z 820.48±0.1, m/z 831.53±0.1, m/z 943.60±0.1, m/z 995.56±0.1, m/z 1032.71±0.1, m/z 1157.68±0.1, m/z 1170.69±0.1, m/z 1349.77±0.1, m/z 1684.94±0.1, m/z 1791.97±0.1, and m/z 1850.01±0.1.

Aspect 40. The method according to one or more of the foregoing aspects, wherein the method has a sensitivity and a specificity of at least 75%, at least 90%, at least 95%, at least 98%, and/or at least 99% in correctly classifying the tissue sample as one of cancerous and non-cancerous.

All documents cited herein are incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to this application.

While particular embodiments have been illustrated and described, it would be obvious to those skilled in the art that various other changes and modifications may be made without departing from the spirit and scope of the invention. Those skilled in the art will recognize, or be able to ascertain using no more than routine experimentation, numerous equivalents to the specific apparatuses and methods described herein, including alternatives, variants, additions, deletions, modifications and substitutions. This application including the appended claims is therefore intended to cover all such changes and modifications that are within the scope of this application.

### CITED REFERENCES

Ferlay J, Soerjomataram I, Ervik M, et al. GLOBOCAN 2012 v1.0, Cancer Incidence and Mortality Worldwide: IARC CancerBase No. 11. Lyon, France: International Agency for Research on Cancer. 2013.
Lagergren J, Bergström R, Lindgren A, Nyrén O. Symptomatic gastroesophageal reflux as a risk factor for esophageal adenocarcinoma. N Engl J Med. 1999; 340(11):825-831.
Kong C Y, Nattinger K J, Hayeck T J, et al. The impact of obesity on the rise in esophageal adenocarcinoma incidence: estimates from a disease simulation model. Cancer Epidemiol Biomarkers Prev. 2011; 20(11):2450-2456.
Walsh T N, Noonan N, Hollywood D, Kelly A, Keeling N, Hennessy T P J. A comparison of multimodal therapy and surgery for esophageal adenocarcinoma. N Engl J Med. 1996; 335(7):462-467.
Tepper J, Krasna M J, Niedzwiecki D, et al. Phase III trial of trimodality therapy with cisplatin, fluorouracil, radiotherapy, and surgery compared with surgery alone for esophageal cancer: CALGB 9781. J Clin Oncol. 2008; 26(7):1086-1092.
van Hagen P, Hulshof M C, van Lanschot J J, et al. Preoperative chemoradiotherapy for esophageal or junctional cancer. N Engl J Med. 2012; 366(22):2074-2084.
Rohatgi P, Swisher S C, Correa A M, et al. Characterization of pathologic complete response after preoperative chemoradiotherapy in carcinoma of the esophagus and outcome after pathologic complete response. Cancer. 2005; 104(11):2365-2372.
Orditura M, Galizia G, Morgillo F, et al. Complete response to preoperative chemoradiation and survival in esophageal cancer: a pooled analysis of three single institution phase II trials. Dis Esophagus. 2012; 25(2):130-136.
Herskovic A, Martz K, Al-Sarraf M, et al. Combined chemotherapy and radiotherapy compared with radiotherapy alone in patients with cancer of the esophagus. N Engl J Med. 1992; 326(24):1593-1598.
Luthra R, Wu T T, Luthra M G, et al. Gene expression profiling of localized esophageal carcinomas: association with pathologic response to preoperative chemoradiation. J Clin Oncol. 2006; 24(2):259-267.
Uemura N, Kondo T. Current status of predictive biomarkers for neoadjuvant therapy in esophageal cancer. World J Gastrointest Pathophysiol. 2014; 5(3):322-334.
Ajani J A, Correa A M, Hofstetter W L, et al. Clinical parameters model for predicting pathologic complete response following preoperative chemoradiation in patients with esophageal cancer. Ann Oncol. 2012; 23(10):2638-2642.
van Rossum P S, Fried D V, Zhang L, et al. The incremental value of subjective and quantitative assessment of 18F-FDG PET for the prediction of pathologic complete response to preoperative chemoradiotherapy in esophageal cancer. J Nucl Med. 2016; 57(5):691-700.

What is claimed is:

1. A method of analyzing a tissue sample comprising:
(a) generating sample ions directly from the tissue sample using a MALDI ionization source;
(b) receiving the ions into a mass spectrometer;
(c) identifying at least one esophageal tumor related compound in the tissue sample from results from the mass spectrometer;
(d) comparing the at least one identified esophageal tumor related compound in the tissue sample to one or more known esophageal tumor profiles; and
(e) identifying at least one condition related to the tissue sample from the comparison of the at least one identified esophageal tumor related compound to the one or more known esophageal tumor profiles.

2. The method of claim 1, wherein the tissue sample comprises an esophageal tumor sample from a subject characterized as at least one of chemoradiation therapy responsive, chemoradiation therapy partially responsive, and chemoradiation therapy nonresponsive.

3. The method of claim 1, wherein the one or more known esophageal tumor profiles comprise at least one of a profile from a plurality of chemoradiation therapy responsive esophageal tumor samples, a profile from a plurality of chemoradiation therapy partially responsive esophageal tumor samples, and a profile from a plurality of chemoradiation therapy nonresponsive esophageal tumor samples.

4. The method of claim 3, wherein the one or more known esophageal tumor profiles comprise at least one of m/z 630.41±0.1, m/z 769.48±0.1, m/z 820.48±0.1, m/z 831.53±0.1, m/z 943.60±0.1, m/z 995.56±0.1, m/z 1032.71±0.1, m/z 1157.68±0.1, m/z 1170.69±0.1, m/z 1349.77±0.1, m/z 1684.94±0.1, m/z 1791.97±0.1, and m/z 1850.01±0.1.

5. The method of claim 1, wherein the at least one condition comprises at least one of chemoradiation therapy responsive, chemoradiation therapy partially responsive, and chemoradiation therapy nonresponsive.

6. The method of claim 1, wherein identifying at least one esophageal tumor related compound in the sample from results from the mass spectrometer comprises generating a mass spectrometry profile of the tissue sample from results from the mass spectrometer.

7. The method of claim 6, wherein the mass spectrometry profile comprises at least one of m/z 630.41±0.1, m/z 769.48±0.1, m/z 820.48±0.1, m/z 831.53±0.1, m/z 943.60±0.1, m/z 995.56±0.1, m/z 1032.71±0.1, m/z 1157.68±0.1, m/z 1170.69±0.1, m/z 1349.77±0.1, m/z 1684.94±0.1, m/z 1791.97±0.1, and m/z 1850.01±0.1.

8. The method of claim 1 characterized by a sensitivity and a specificity of at least 95% in correctly classifying the tissue sample as one of cancerous and non-cancerous.

9. The method of claim 1 comprising staining the tissue sample prior to generating sample ions directly from the tissue sample using a MALDI ionization source.

10. The method of claim 1 comprising staining the tissue sample after generating sample ions directly from the tissue sample using a MALDI ionization source.

11. The method of claim 1 comprising staining the sample with hematoxylin and eosin.

12. The method of claim 1 comprising:
treating the tissue sample to deparaffinization and antigen retrieval prior to on-tissue tryptic digestion and matrix application using a robotic sprayer;
treating the tissue sample to on-tissue tryptic digestion; and
applying a MALDI compatible matrix to the treated tissue sample using a robotic sprayer.

13. The method of claim 1, wherein the tissue sample comprises a first section and a second section, and wherein the method comprising:
annotating an image of the first section to identify areas of interest on the tissue sample having a diameter from 10-400 micrometers; and
imposing the annotated image of the first section upon the second section prior to generating sample ions directly from the areas of interest identified on the second section of the tissue sample using the MALDI ionization source.

14. The method of claim 13, wherein the areas of interest are identified as one of cancerous and non-cancerous using histologic analysis and/or immunohistochemical analysis.

15. The method of claim 1, wherein the tissue sample is obtained from a patient.

16. The method of claim 14 comprising treating the patient with at least one of chemotherapy, immunotherapy, toxin therapy, surgery, and radiotherapy when the patient is identified as having at least one of a chemoradiation therapy responsive condition, a chemoradiation therapy partially responsive condition, and a chemoradiation therapy nonresponsive condition.

17. A method of treating an esophageal tumor in the patient comprising:
(i) determining a chemoradiation responsive condition in a patient by
(a) obtaining a tissue sample from the patient;
(b) generating sample ions directly from the patient's sample using a MALDI ionization source;
(c) receiving the ions into a mass spectrometer;
(d) identifying at least one esophageal tumor related compound in the tissue sample from results from the mass spectrometer; and
(ii) administering at least one of chemotherapy, immunotherapy, toxin therapy, surgery, and radiotherapy to the patient to treat the esophageal tumor when the patient is identified as one of chemoradiation therapy responsive, chemoradiation therapy partially responsive and chemoradiation therapy nonresponsive.

18. The method of claim 17 comprising:
(e) comparing the at least one identified esophageal tumor related compound in the tissue sample to one or more known esophageal tumor profiles; and
(f) identifying at least one condition related to the tissue sample from the comparison of the at least one identified esophageal tumor related compound to the one or more known esophageal tumor profiles.

19. The method of claim 17, wherein the one or more known esophageal tumor profiles comprise at least one of m/z 630.41±0.1, m/z 769.48±0.1, m/z 820.48±0.1, m/z 831.53±0.1, m/z 943.60±0.1, m/z 995.56±0.1, m/z 1032.71±0.1, m/z 1157.68±0.1, m/z 1170.69±0.1, m/z 1349.77±0.1, m/z 1684.94±0.1, m/z 1791.97±0.1, and m/z 1850.01±0.1.

20. The method of claim 17, wherein the method has a sensitivity and a specificity of at least 75% in correctly classifying the tissue sample as one of cancerous and non-cancerous.
